(19) United States
(12) Patent Application Publication
(10) Pub. No.: US 2014/0135642 A1
(43) Pub. Date: May 15, 2014

(54) NATURE-INSPIRED SIGNAL PROCESSING

(71) Applicant: FRANK EDUGHOM EKPAR, Aizuwakamatsu City (JP)

(72) Inventor: FRANK EDUGHOM EKPAR, Aizuwakamatsu City (JP)

(21) Appl. No.: 13/674,035

(22) Filed: Nov. 11, 2012

(51) Int. Cl. A61B 5/0476; A61F 4/00; G06F 3/01

(52) U.S. Cl. CPC A61B 5/0476; G06F 3/015; A61F 4/00
USPC 600/544; 607/2; 607/62

(57) ABSTRACT

The present invention establishes the foundational principles and practice for a unified theory of arbitrary information management by disclosing systems, devices and methods for the management of substrates or biological substrates. In this context, a substrate or biological substrate is any aspect of any entity that is capable of responding to or emitting or transmitting stimuli irrespective of whether the stimuli actually emanate or originate from any aspect of the entity or not. Management of substrates or biological substrates could be achieved through the management of stimuli that characterize, modulate or moderate or influence any aspect of the substrate or biological substrate as well as through the management of any stimuli emanating from the substrate or biological substrate.

### CROSS-REFERENCE TO RELATED APPLICATIONS

This United States (U.S.) Non-Provisional application claims the benefit of U.S. Provisional Application Ser. No. 61/580,202, filed on Dec. 24, 2011, herein incorporated by reference.

### BACKGROUND OF THE INVENTION

1. Field of the Invention

The present invention relates generally to the field of nature-inspired bio-signal processing and allied fields. Systems, devices and methods for the management of biological substrates are disclosed. Narrowly in this context, a biological substrate is any aspect of any biological system such as any aspect of any actual biological entity or living organism or any simulation of any biological entity as in a computer system or computer-implemented system or any artificial system with characteristics approximating those of any actual or conceptualized biological entity. Generally in this context, a biological substrate is any aspect of any entity that is capable of responding to or emitting or transmitting stimuli irrespective of whether the stimuli actually emanate or originate from any aspect of the entity or not. Stimuli reflected from or otherwise detectable from any aspect of the entity could be taken into consideration. Similarly, indirect responses to stimuli that could be associated with any aspect of the entity could also be taken into consideration. Consequently, any aspect of any entity including any aspect of any animate or inanimate entity that is responsive to stimuli or that emits or transmits stimuli or that does both is a biological substrate within the context of the present invention. The term “biological substrate” has been adopted to emphasize the far-reaching implications of the present invention to biological systems and does not limit the scope of the present invention to biological systems. Management of biological substrates could be achieved through the management of stimuli that characterize, modulate or moderate or influence any aspect of the biological substrate as well as through the management of any stimuli emanating from the biological substrate. More specifically, systems, devices and methods for the generation, acquisition, processing, storage, distribution and utilization of bio-signals or stimuli related to biological substrates that fall within the scope of this invention are disclosed. In one embodiment, a bio-signal acquisition or generation device comprising a grid of sensor or transducer ensembles wherein each sensor or transducer ensemble comprises one or more sensors or transducers disposed on an arbitrarily shaped and arbitrarily sized N-dimensional surface wherein each sensor or transducer is connected operatively to a suitable medium—such as a conducting medium in the case of bio-signals of an electrical or electrochemical nature—which in turn is connected operatively to a surface associated with the source of the bio-signals wherein the sensors or transducers are responsive to (or generate in the case of actuators) stimuli from (in the case of sensors) or within (in the case of actuators) the biological substrate with the assembly thus comprising a counterpart environment to the target or underlying biological substrate is used to acquire or generate bio-signals. Furthermore, the invention relates to methods of increasing the information transfer rate (measured in bits per second: the product of information transfer per presentation—in bits per item—and the presentation rate—in items per second) of brain-computer interface systems.

2. Description of the Prior Art

Biological systems such as plants and animals are associated with a wide variety of signals. In this context, these signals are referred to as bio-signals and are understood to include both intrinsic signals generated by the biological system for its own purposes and extrinsic signals that can be used to manipulate selected aspects of the biological system. Plants are known to communicate using chemical signals. Ian T. Baldwin and Jack C. Schultz [1] report evidence for communication between plants mediated by phenolic compounds and suggest that an airborne cue generated by damaged plant tissue may elicit biochemical changes in neighboring plants that could have an impact on the feeding and growth of phytophagous (plant-eating) insects. In essence, damaged plants seem to generate signals that are conducive to their survival as well as induce the generation of similar signals in neighboring plants. R. Karban et. al. [2] demonstrate rigorous experimental evidence for induced resistance to herbivores in wild tobacco plants following the clipping of neighboring sagebrush, reporting that wild tobacco plants with clipped sagebrush neighbors exhibited higher levels of the putative defensive oxidative enzyme polyphenol and experienced significantly reduced leaf damage from grasshoppers and cutworms than controls. Echo-locating bats have been reported to use sonar for navigation and foraging [3-7].

Sharks and related species of fish use electro-sensory structures known as ampullae of Lorenzini for the localization of prey [8-12]. The artificial pacemaker and defibrillator are well known examples of the application of electrophysiological signals associated with the human heart [13-17]. Ultimately, in many biological systems found in nature—including the human body—, bio-signals are generated in, mediated by, and exert their influence on intracellular and/or extracellular (in the case of multi-cellular systems) structures and processes. In this regard, many studies of the electrophysiological and related characteristics of intracellular and extracellular structures and tissues have been reported in the literature and include the Hodgkin-Huxley model that gives a theoretical description of excitable membrane. The patch clamp method was introduced by Edwin Neher and Bert Sakmann and permitted the measurement of the membrane current of a single ion channel. Further refinement of the patch clamp technique allowed the determination of cell membrane capacitance and subsequently led to studies harnessing minute changes in membrane surface area to characterize secretory processes [18-34].

A wide variety of modalities are harnessed in the study of bio-signals associated with the human brain. These include, but are not limited to, positron emission tomography (PET) [35], single-photon emission computed tomography (SPECT), electroencephalography (EEG), electrocorticography (EcoG), magneto-encephalography (MEG), functional magnetic resonance imaging (fMRI) and functional near-infrared spectroscopy (fNIR). Each of these modalities has its merits and demerits when compared with the others. Electroencephalograph (EEG) and magneto-encephalography (MEG) are remarkable in the sense that the EEG signal originates from the electrical activity of neuronal populations and can be measured directly using simple electrodes. Similarly, based on Maxwell's equations, the electrical activity of neuronal populations yields a magnetic field that can be measured directly using a magnetometer in MEG [36, 37]. This is not the case for other modalities that rely on indirect measures of brain activity. Modern MEG devices typically utilize ultrasensitive superconducting quantum interference devices (SQUIDS) [38] arrays for the detection of the weak magnetic fields that originate from the brain's electrical activity. The EEG has is a non-invasive technique (involving the placement of electrodes or sensors on the scalp), can be implemented at relatively low cost, imposes fewer restraints on the movement of the subject (allowing longer duration recordings) and has good temporal resolution—in the order of 1 millisecond—but is hampered by low spatial resolution. The optimum electrode distance for the EEG seems to be between 10.0 mm and 50.0 mm on the basis of estimated variable brain-skull-scalp resistivity ratios and the use of the reciprocity theorem, superposition principle, lead field theorem and theoretical spatial frequency (spatial Nyquist) considerations [39-44]. Attempts to localize the sources of the EEG signals (or to solve the forward and inverse problems) typically employ large numbers of electrodes (in the order of 100 electrodes) and provide only crude estimates [45-47]. Researchers have demonstrated the feasibility of brain-computer interfaces (BCIs) based solely on noninvasively acquired EEG signals [48]. Generally, contemporary noninvasive EEG measurement systems utilize electrodes placed on the scalp with a single electrode or sensor per site. Thus for each site on the scalp, only a single signal stream—presumably representing the superposition of signal contributions of layers of neuronal populations beneath the site—is typically acquired. However, the simultaneous acquisition of distinct signals representing the contributions of different layers of neuronal populations by multiple electrodes at the same site could significantly improve the spatial resolution of the EEG, provide new insights into the underlying physiological processes and open up new avenues for the application of the EEG.

It is clear from the foregoing that the characterization as well as the modulation or moderation or control as well as the generation of stimuli related to biological systems (and more generally biological substrates as defined in the context of the present invention) provide significant opportunities for the furthering of human understanding of these systems and consequently offer significant new avenues for the beneficial application of such systems in wide application areas ranging from brain-computer or brain-machine interfaces to the diagnosis and treatment of many diseases, illnesses and medical conditions as well as vast new arenas in entertainment and other useful applications. For simplicity however, the embodiments disclosed in the present invention will focus on the brain (and especially the human brain as well as the brains of other animals such as primates) and related applications.

The functional organization of many regions of the brain including the superior temporal cortex which is believed to play a critical role in the hierarchical processing of human visual and auditory stimuli is not well understood. It is not known precisely which layer within which region of the brain is responsible for which aspect of visual or auditory processing. Simultaneous non-invasive acquisition of bio-signals representing contributions from multiple layers of neuronal populations within the brain could provide new insights leading to the resolution of many of these outstanding issues and provide a deeper understanding of the underlying physiological processes. However, the simultaneous acquisition of bio-signals from multiple layers within the signal source at sufficient temporal and spatial resolution to resolve these and other critical questions is impracticable using state-of-the-art non-invasive bio-signal acquisition systems such as electroencephalography (EEG) and magnetic resonance imaging (MRI) that are routinely used in the diagnosis and treatment of diseases as well as for research aimed at understanding the neurological underpinnings of human and animal behavior.

It has been shown that elasmobranchs—a scientific grouping containing fish such as sharks, rays, and skates possess bio-signal sensing electroreceptors named ampullae of Lorenzini which they utilize for such purposes as the location of prey Ampullae of Lorenzini are electroreceptive units in elasmobranchs comprising jelly-filled canals found on the head of the animal which form a system of sense organs, each of which receives stimuli from the outside environment through the dermis and epidermis. Each canal ends in groups of small bulges lined by the sensory epithelium. A small bundle of afferent nerve fibers innervates each ampullae. Although the lengths of the canals vary from species to species—even within any one fish—, the pattern of distribution is approximately species specific. (Murray, R. W. 1974. The Ampullae of Lorenzini. In: Handbook of Sensory Physiology., A. Fessard, (ed). Springer-Verlag, New York.).

Neuronal populations in human and animal brains generate signals which can be used to drive brain-computer interfaces.

Brain-computer interfaces (BCI) are systems that serve as communication pathways between humans (and generally animals) and machines. In BCIs, signals corresponding directly or indirectly to physiological and cognitive processes in the subject could be translated into commands that could be used to control external devices. Conversely, signals from external sensors could be transformed into a suitable format and used to induce perceptions in the subject that would ordinarily be induced through the normal operation of the body's natural sensory organs. Thus, BCIs provide means of circumventing the usual motor-sensory pathways in the subject and could be harnessed as an independent channel of communication with the subject's environment. For subjects with impairments, the circumvention of the traditional motor-sensory pathways facilitated by BCIs hold the promise of a viable means of restoring interaction with the environment that would otherwise be impossible or difficult to attain. Healthy subjects could also use BCIs as alternative and potentially more intuitive communication channels.

A variety of methods and devices—each with its own set of advantages and drawbacks—can be used to acquire brainwave data. These generally fall into two broad categories—invasive and non-invasive. Invasive methods and systems are characterized by the utilization of intra-cranial means of recording signals while non-invasive methods and systems typically involve the measurement of signals without direct contact with the cells generating the signals. The electrocorticography (ECoG) technique described by Leuthardt; Eric C. et al. in U.S. Pat. No. 7,120,486 involves the recording of the electrical activity of the cerebral cortex by means of electrodes placed directly on it, either under the dura mater (subdural) or over the dura mater (epidural) but beneath the skull and is thus an example of an invasive method of brainwave signal acquisition. Systems based on functional magnetic resonance imaging (fMRI), positron emission tomography (PET), single photon emission computerized tomography (SPECT), electroencephalography (EEG), magnetoencephalography (MEG), and functional near-infrared spectroscopy (fNIRS) provide non-invasive means of brainwave recording and depend on a variety of principles ranging from neurovascular coupling (the relationship between blood flow in neural cell populations and cognitive activity involving the participation of said neural cell populations) to electrophysiological analyses. Invasive techniques generally provide more accurate representations of neuronal activity but are hampered by the associated risks and inconvenience of brain surgery (for implantation of the recording device) and degeneration of signal quality due to encapsulation of the recording electrodes by fibrous tissue and/or destruction of neighboring cells by the electrodes.

Currently, the majority of non-invasive BCIs are based on the well-known electroencephalography (EEG) technique owing to its relative portability, low cost, high temporal resolution and ease of operation. Examples of BCIs based on EEG and/or other non-invasive recordings include those disclosed in U.S. Pat. No. 5,638,826, U.S. Pat. No. 7,403,815 and U.S. Pat. No. 6,349,231. The spatial resolution of contemporary EEG-based BCIs is quite low—with systems typically comprising between 1 and 256 electrodes, each of which aggregates signals from massive neuronal populations. Furthermore, the signals are heavily attenuated on their journey through the skull and are thus susceptible to corruption by noise from other signal-emitting physiological processes in the subject and disturbances from the environment.

Techniques, algorithms and systems that remedy the shortcomings of EEG-based BCIs are well known and widely reported in the literature. Writing in the Proceedings of the United States National Academy of Sciences (2004 Dec. 21; 101(51): 17849-17854), Jonathan R. Wolpaw and Dennis J. McFarland describe an adaptive algorithm that uses a simple linear combination of relevant features to improve the effectiveness of a non-invasive BCI designed for 2-dimensional computer cursor control. Although the method described by Jonathan R. Wolpaw et al. provides better results than some competing methods by adapting the features selected for classification to the specific features that the user is best able to control, it is still hampered by the major drawback of high sensitivity to individual brainwave characteristics and the requirement for long training periods. The information transfer rate of EEG-based BCIs is currently in the range of 5 to 25 bits per second which is too low to permit widespread use of such BCIs in practical applications.

Deep brain stimulation encompasses a wide range of well-known techniques for the generation and utilization of electrical or other impulses, signals or stimuli that are applied to selected regions of the brain to produce desired states and outcomes. State of the art deep brain stimulation practice typically involves the invasive surgical implantation of electrodes (for the generation of electrical stimuli or impulses or signals) or other devices such as brain pacemakers with the attendant drawbacks in terms of long-term viability, undesirable side effects and limited efficacy. For example, U.S. Pat. No. 8,295,935 discloses new methods for deep brain stimulation (DBS) surgery using two or more electrical leads that are surgically implanted in the brain of the patient. Other deep brain stimulation (DBS) modalities such as the application of light energy as described in U.S. Pat. No. 8,303,636 that prescribes trans-cranially applying light energy having a wavelength of between 300 nm to 1500 nm and a power density at the scalp of up to 320 mW per square cm to the brain of a patient with anxiety disorder with hemispheric asymmetry and similar applications deep brain stimulation (DBS) are also available. These contemporary applications are all limited by the lack of precision in the targeting of brain regions since these contemporary systems are unable to simultaneously and precisely target specific layers within the brain for better clinical outcomes and generally significantly better results. Where contemporary systems are able to achieve targeting of specific brain regions, they are typically limited to the modulation or moderation or control or stimulation of just one imprecisely defined region at a time.

### SUMMARY OF THE INVENTION

It is an object of the present invention to overcome the limitations of the prior art set forth above by providing improved systems, devices and methods for the management of biological substrates. According to the principles of the present invention, management of biological substrates could be achieved through the management of stimuli that characterize, modulate or moderate or influence or control any aspect of the biological substrate as well as through the management of any stimuli emanating from the biological substrate. More specifically, systems, devices and methods for the generation, acquisition, processing, storage, distribution and utilization of bio-signals or stimuli related to biological substrates that fall within the scope of the present invention are disclosed.

In one embodiment, a bio-signal acquisition or generation device comprising a grid of sensor or transducer ensembles wherein each sensor or transducer ensemble comprises one or more sensors or transducers disposed on an arbitrarily shaped and arbitrarily sized N-dimensional surface wherein each sensor or transducer is connected operatively to a suitable medium—such as a conducting medium in the case of bio-signals of an electrical or electrochemical nature—which in turn is connected operatively to a surface associated with the source or target of the bio-signals (biological substrate) wherein the sensors or transducers are responsive to (or generate in the case of actuators) stimuli from (in the case of sensors) or within (in the case of actuators) the biological substrate with the assembly thus comprising a counterpart or corresponding environment to the target or underlying biological substrate is used to acquire or generate bio-signals. Such systems permit the simultaneous non-invasive acquisition of one or more distinct bio-signal streams or stimuli from the same site using a sensor ensemble. The systems permitted by the principles of the present invention also enable the efficient and non-invasive generation of one or more distinct bio-signal streams or stimuli at the same site using an actuator ensemble. N can be 1 (for linear embodiments), 2 (for planar embodiments), 3 (for conventional spatial or 3-dimensional embodiments), 4 (for time-varying spatial or 3-dimensional embodiments), or any other suitable number of dimensions required for a given application or set of applications.

The results permitted by the present invention have far-reaching implications for many application domains—such as the clarification of the functional organization of critical regions of the brain—in which the simultaneous acquisition of bio-signals or stimuli from multiple layers could lead to better understanding and more efficient operation.

Furthermore, the results permitted by the present invention have far-reaching implications for many application domains—such as the use of generated stimuli for the modulation or stimulation or moderate or control of selected regions of a biological substrate—in which the ability to simultaneously modulate or stimulate or moderate or control multiple layers within a biological substrate could lead to better understanding, better diagnosis and treatment outcomes and more efficient operation.

Additionally, the ability of the present invention to permit simultaneous acquisition of multiple streams of stimuli from a sensor site and the generation of stimuli streams that can simultaneously modulate or moderate or control or stimulate multiple layers within a biological substrate leads to vastly improved spatial resolution as well as vast increases in the information transfer rate (measured in bits per second: the product of information transfer per presentation—in bits per item—and the presentation rate—in items per second) of brain-computer interface systems or similar systems based on the principles of the present invention.

### BRIEF DESCRIPTION OF THE DRAWINGS

FIG. 1 shows a representative model describing aspects of the principles of the present invention.

FIG. 2 depicts how the representative model describing aspects of the principles of the present invention could be applied to stimuli related to the brain.

FIG. 3 illustrates a vertical cross-section through a representative sensor ensemble.

FIG. 4 depicts a simple alternative sensor ensemble.

FIG. 5 shows a top view illustrating the partitioning of the containing cylinder into unique planes. P1 and P2 represent the outlines of two planes. The angle between them is θ.

FIG. 6 illustrates a 3-dimensional view of the containing cylinder showing two representative planar partitions P1 and P2 separated by the angle θ.

FIG. 7 shows a subdivision of a plane into m strips. Transducers e1, e2, . . . , em are embedded in the strips, one transducer per strip.

FIG. 8 illustrates planar partitions P1, P2, . . . , Pn with embedded transducers SE1, SE2, . . . , SE3.

FIG. 9 depicts a double helix formed by partitioning the containing cylinder into unique planes and embedding a single transducer per plane at a distinct height from the base of the cylinder.

FIG. 10 illustrates the splitting of a transducer on a plane P1 into two opposing transducers SE1 and SE2 at a height d12 from the base of the cylinder and the double-stranded double helix formed by partitioning the containing cylinder into unique planes and embedding two opposing and horizontally separated transducers per plane at a distinct height from the base of the cylinder.

FIG. 11 depicts tri-linear interpolation for a site bounded by eight neighboring transducer sites in a 3-dimensional spatial configuration.

FIG. 12 shows a flowchart outlining how brainwave signals could be processed according to the principles of the present invention.

FIG. 13 illustrates the tier-1 image representation used by the preferred embodiment of the present invention.

FIG. 14 shows the partitioning or segmentation of the original image frame to form tier-2 of the image representation used by the preferred embodiment of the present invention.

### DETAILED DESCRIPTION OF THE PREFERRED EMBODIMENTS

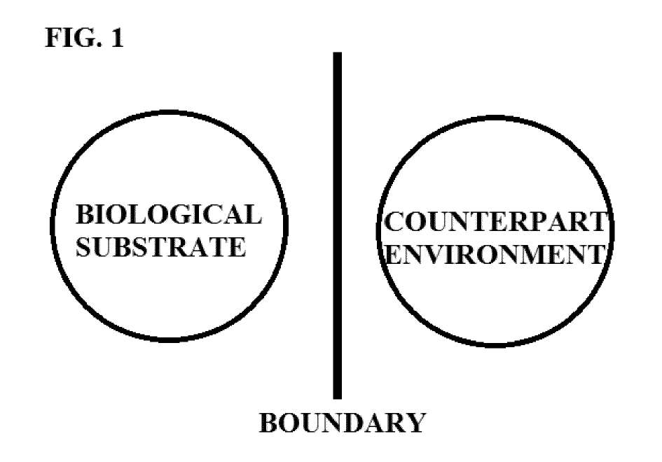

In FIG. 1, a representative model describing aspects of the principles of the present invention shows a biological substrate—labeled BIOLOGICAL SUBSTRATE—and a counterpart or corresponding environment—labeled COUNTERPART ENVIRONMENT—separated by a boundary—labeled BOUNDARY.

This model of aspects of the principles of the present invention is one in which approximations to carefully selected representative features of the source or target of the bio-signals or biological substrate are utilized for characterization and/or manipulation or modulation or moderation or control or stimulation of the underlying biological system.

An environment corresponding to selected features of the source or target biological substrate is created and a strategy that utilizes the corresponding or counterpart environment for the characterization and/or manipulation or modulation or moderation or control or stimulation of the underlying biological system is pursued.

The choice of features to approximate in the corresponding or counterpart environment depends on the application of the model. Similarly, the characteristics of the boundary between the source or target of the bio-signals or biological substrate and the corresponding or counterpart environment created in the model depend on the features of the source or target and the corresponding or counterpart environment.

An operative connection between the counterpart environment and the biological substrate is also established. This connection could be as simple as merely placing the counterpart environment in physical (or other suitable operative) contact with the biological substrate. In the case where the biological substrate is the brain, the operative connection between the counterpart environment comprising the transducers (sensors or actuators) and related systems and the biological substrate comprising the brain tissue could be the placement of a conducting medium such as a conducting gel in contact with the skull of the brain which in this case forms the boundary between the counterpart environment and the biological substrate. One of ordinary skill in the art will appreciate that the operative connection need not involve physical contact between the counterpart environment and the biological substrate but could comprise the air or some other gap or any other suitable interface between the two environments such as a lens or other focusing element in applications requiring the use of light or optical signals or sound or microwave signals for operation. Suitably formatted or adapted interfaces (including shielding when needed) could be applied in applications requiring the use of magnetometers or similar devices or systems for operation.

According to the principles of the present invention, the biological substrate could be used to influence or modulate or moderate or control the counterpart environment. This could be achieved by designing and connecting the counterpart environment to the biological substrate in such a way that stimuli that are emitted by or transmitted from the biological substrate influence or modulate or moderate or control aspects of the counterpart environment. Conversely, the counterpart environment could be used to influence or modulate or moderate or control the biological substrate. This could be achieved by designing and connecting the counterpart environment to the biological substrate in such a way that stimuli that are emitted by or transmitted from the counterpart environment influence or modulate or moderate or control aspects of the biological substrate.

It should be obvious to one of ordinary skill in the art that this representative model is by no means the only model that could be used to explain or implement aspects of the principles of the present invention. In fact this model need not be correct in a rigorous sense but is intended only as a guide for the explanation and implementation of aspects of the principles of the present invention. Other suitable models may be applied without deviating from the principles of the present invention.

Characterization of a biological substrate could involve the determination of the state of selected aspects of the biological substrate. For applications related to the brain, this could involve the sensing of stimuli associated with the state of excitation or relaxation or other relevant state of selected neuronal populations. This state information could be collected using electrodes, magnetometers or any other suitable systems, methods or devices.

Numerous techniques for the mathematical, visual or other suitable representation or characterization of the information associated with biological substrates exist in the relevant literature and some of these have already been cited in the description of the prior art.

Similarly, modulation or moderation or control or stimulation of a biological substrate could involve effecting or causing a desired state in any selected aspects of the biological substrate. For applications related to the brain, this could involve the use of suitable stimuli such as electrical impulses, magnetic impulses, light waves, radio signals or any other suitable stimuli to cause or effect the excitation or relaxation or other relevant state of selected neuronal populations. This modulation or moderation or control or stimulation could be effected or caused using electrodes, magnetometers or any other suitable systems, methods or devices.

Means of achieving modulation or moderation or control or stimulation of a biological substrate include deep brain stimulation and other techniques reported in the relevant literature, some of which have already been cited in the description of the prior art.

As explained earlier, the created counterpart or corresponding environment could comprise one or more transducer (sensor or actuator) ensembles with each ensemble containing one or more transducers (sensors or actuators) and the assembly disposed on an arbitrarily shaped and arbitrarily sized N-dimensional surface wherein each transducer (sensor or actuator) is connected operatively to a suitable medium—such as a conducting medium in the case of bio-signals of an electrical or electrochemical nature—which in turn is connected operatively to a surface associated with the biological substrate (such as the skull in the case of applications related to the brain) wherein the transducers are responsive to (in the case of sensors) or generate (in the case of actuators) stimuli from (in the case of sensors) or within (in the case of actuators) the biological substrate. One of ordinary skill in the art will appreciate that it is possible to configure any of the elements or transducers in any of the ensembles as a sensor responsive to suitable stimuli or as an actuator generating suitable stimuli or both as a sensor responsive to suitable stimuli as required and as an actuator generating suitable stimuli as required. Furthermore, applications in which the characterization of stimuli from a biological substrate based on information collected from one or more sensors is utilized as a guide for the modulation or moderation or control or stimulation of the biological substrate fall within the scope of the present invention. Such applications include aptitude or similar training scenarios in which the characterization of a biological substrate could be used to provide feedback for the modulation or moderation or control or stimulation of the biological substrate.

This representative model disclosed in the present or any other suitable model based on the principles of the present invention could be used to simulate any desired aspects of a biological substrate on any suitable system such as a computer system and associated display systems.

Information gleaned from sensors responsive to stimuli emanating from a biological substrate could be suitably formatted and displayed in a manner that adequately characterizes any desired aspects of the biological substrate. For example, information from electrodes representing the state of excitation or relaxation of selected neuronal populations could be formatted and displayed on a suitable computer display as a color map that would make it easy to determine whether the subject the biological substrate represents is excited, depressed, agitated or in some other relevant emotional state. Such information could also be harnessed by using suitable systems such as computer-implemented software code written in a suitable programming language such as C, C++, JAVA, Python, and so on, to provide commands for the control of other systems or devices such as software running on the same computer systems or other computer systems connected to the computer system on which the processing takes place through the Internet or some other suitable network. It should be obvious to one of ordinary skill in the art that a wide variety of applications can be created using information gleaned from the created counterpart environment based on the principles of the present invention.

Similarly, a wide variety of applications can be created by controlling or manipulating the created counterpart environment in a manner that facilitates directed and purposeful modulation or moderation or control or stimulation of a biological substrate in accordance with the principles of the present invention. For example, more precise and significantly more useful deep brain stimulation can be achieved through the precise targeting of relevant brain layers or regions for stimulation in accordance with the principles of the present invention.

It should be noted that any aspect of the created counterpart or corresponding environment may be employed towards the characterization and modulation or moderation or control or stimulation of the biological substrate. These aspects include, but are not limited to, interactions and consequences of interactions between the elements of the created counterpart or corresponding environment themselves. In fact, such interactions could be purposefully designed and exploited to better characterize and modulate or moderate or control or stimulate the biological substrate.

Consider the situation where the source of the bio-signals is the human brain and the goal is the acquisition of the corresponding electroencephalography (EEG) signals with minimal or no corruption from the distortions introduced by the brain tissue, skull and scalp. Then based on this model, an environment (counterpart or corresponding environment) could be created outside the brain—with a boundary comprising a conducting medium in contact with the scalp and the corresponding environment—in which approximations to the distortions are used to inform the placement of sensors in such a manner as to mitigate or eliminate the effects of the distortions. Approaches such as spatial de-convolution could be used to correct distortions in a post-acquisition step [49] but it would be more convenient to mitigate the effect of such distortions at the signal acquisition stage. It should be noted that although a conducting medium is suitable in this case, the model places no restrictions on the characteristics of the medium—any suitable medium can be employed based on the requirements of the specific application of the model.

Now suppose the goal is to simultaneously acquire the EEG from different depths within the brain. In this case an environment—outside the brain—that approximates the propagation delays as the signals propagate from sources within the brain to the scalp could be created and sensors could be positioned in the corresponding environment in a manner that would allow the simultaneous acquisition of signals from different depths within the brain. Here the boundary could also comprise a conducting medium in contact with the scalp and the corresponding environment.

As already explained, the representative model proposed here and any predictions based on the representative model need not be correct. Numerous alternative models could be proposed and utilized in practicing the present invention.

In particular, the following predictions based on the proposed model need not be correct. These predictions merely represent a specific interpretation or a specific set of interpretations of the proposed model and should be evaluated for suitability in any given specific application of the present invention.

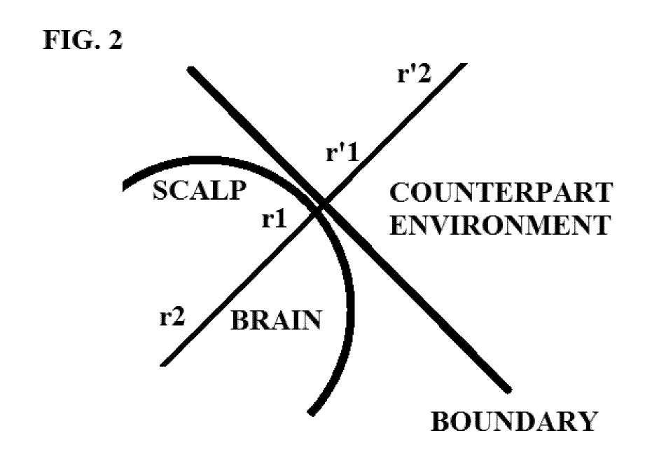

FIG. 2 depicts how the representative model describing aspects of the principles of the present invention could be applied to stimuli related to the brain. In FIG. 2, the boundary—labeled BOUNDARY—between the counterpart environment—labeled COUNTERPART ENVIRONMENT—and the biological substrate (labeled BRAIN) comprises the skull or scalp—labeled SCALP—and the interface (possibly comprising a conducting medium and housing or casing for sensors) between the created counterpart environment and the brain.

Referring now to FIG. 2 in which distances from the boundary in a radial direction from the scalp are depicted, the following predictions can be made based on this model:
I. Since signals from sources at locations deeper in the brain are likely to reach the scalp later than signals originating from neuronal populations or brain regions closer to the scalp, sensors in the corresponding environment closer to the boundary (in a radial direction from the scalp) are likely to detect signals in which contributions from neuronal populations at deeper locations within the brain predominate. Conversely, sensors located farther from the boundary are likely to detect signals in which the contributions of neuronal populations that are closer to the boundary predominate. Referring to the illustration in FIG. 2, a sensor located at r′1 is likely to detect signals in which the contributions of neuronal populations closer to r2 predominate while a sensor located at r′2 is likely to detect signals in which the contributions of neuronal populations closer to r1 predominate.
II. Sensors placed over the same site but separated from each other (in a radial direction from the scalp, such as sensors located at r′1 and r′2 in FIG. 2) are likely to detect signals in which contributions from different levels within the brain predominate.
III. Sensors embedded directly at different levels within the brain should detect signals similar to those detected by sensors located at corresponding positions within the corresponding environment.

The first prediction (Prediction I) may be justified on the basis of the laws of electromagnetic wave propagation as the signals responsible for the EEG are electrical in nature and induce a corresponding magnetic field. Alternative predictions or explanations based on known principles of bio-electromagnetism as described in the relevant literature could also be proposed.

One way to verify or test and modify or discard Prediction III if necessary would be to surgically implant sensors at different depths within the brain and compare the signals acquired with the signals acquired at corresponding locations within the corresponding environment. This would be an invasive procedure.

Prediction II can be verified or tested and modified or discarded if necessary by demonstrating the acquisition of distinct signals from two sensors located at the same site on the scalp. The ampullae of Lorenzini-inspired bio-signal acquisition system described in the present invention could be used to provide just such a demonstration.

### Ampullae of Lorenzini-Inspired Bio-Signal Acquisition System

This section introduces a bio-signal acquisition system inspired by Ampullae of Lorenzini and based on the model described here that harnesses approximations to propagation delays as signals traverse the source to reach the boundary and ultimately—via electrical or electrochemical or other suitable conduction mechanism—the created counterpart or corresponding environment comprising sensors and related systems to facilitate the simultaneous acquisition of signals from different layers within the source. The bio-signal acquisition system comprises a grid of sensor ensembles, each sensor ensemble comprising a collection of sensors disposed on an arbitrarily shaped N-dimensional (N=1, 2, 3, 4, and so on) surface with each sensor in contact with a conducting medium which in turn is in contact with a surface associated with the source of the bio-signals.

Ampullae of Lorenzini are electroreceptive units in elasmobranchs comprising jelly-filled canals found on the head of the animal which form a system of sense organs, each of which receives stimuli from the outside environment through the dermis and epidermis. Each canal ends in groups of small bulges lined by the sensory epithelium. A small bundle of afferent nerve fibers innervates each ampulla. Although the lengths of the canals vary from species to species (even within any one fish), the pattern of distribution is approximately species specific [8-12].

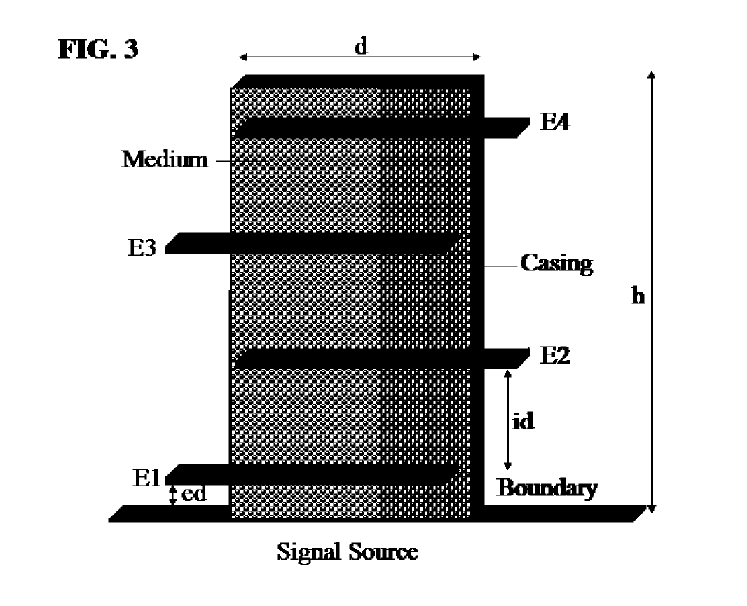

FIG. 3 depicts a representative sensor ensemble with four separate co-planar electrodes labeled E1, E2, E3 and E4 and contained in a plastic (non-conducting) cylindrical casing with a diameter (d) of 10.0 mm and a height (h) of 37.0 mm. The cylinder could be filled with a saline (NaCl)-soaked sponge medium or any other suitable medium and each of the electrodes could be a stainless steel wire with a diameter of about 1.1 mm. The inter-electrode distance (id) could be 5.0 mm and the same for all four electrodes while the distance between the first or base electrode E1 and the boundary between the sensor ensemble and the signal source surface or scalp (ed) could be 7.0 mm. For EEG data acquisition, each electrode in the ensemble could be connected to one electrode—replacing the original sensor—on a suitable EEG headset or any other suitable system. The signal source or biological substrate in this case would be the brain of a suitable subject.

Canals in the ampullae could correspond to the conducting medium in FIG. 3. The sensor casing, housing or support could correspond to the sensor-containing basal region or alveoli of the ampullae. The walls of the sensor-containing basal region or alveoli of the ampullae are typically composed of high resistive or non-conducting material—as is the wall of the sensor casing in FIG. 3. As is generally the case with naturally-occurring ampullae, each ampulla contains a plurality of sensors that could be arranged in an omni-directional fashion for optimum signal coverage. It should be noted that arbitrary configurations of sensor elements are permitted by this model so such omni-directional sensor topologies could also be employed where practicable. The jelly or hydrogel that fills the canals found in ampullae could correspond to the conducting medium—a saline (NaCl)-soaked sponge in this case. As with the sensor topology, the choice of medium depends on the requirements of the specific application of the model.

The characteristics of the individual sensors such as shape, size, material and any other relevant characteristics can be chosen to match individual applications. For example, suitably-shaped gold-plated conductors could be used as electrodes or sensors with a designated sensor at a designated signal source site providing a reference signal. Alternatively, a suitable ground signal or any other suitable signal could be selected as a reference signal. Similarly, the arrangement of sensors or electrodes could be chosen to match individual applications. The sensors or electrodes could be arranged in a 1-, 2-, 3-dimensional or arbitrary configuration as desired. FIG. 3 shows a cross-section representing a 2-dimensional sensor arrangement.

The electrodes could be connected to a signal processing unit similar to those used in contemporary EEG systems for amplification, noise cancellation and/or any other desired processing. In particular, the electrodes and processing unit could be housed in a compact unit such as a suitably designed headband—possibly incorporating a suitable power unit such as a battery and circuitry for wireless transmission of the signals if required. When signals are transmitted wirelessly, a receiver or transceiver (the receiver could conceivably send signals back to the transmitting unit or headband which could also be equipped with a receiver to facilitate bi-directional communication between the units when required) could be connected to a computer or any other suitable device for further processing of the signals.

U.S. Pat. No. 7,567,274 describes a versatile image acquisition device comprising at least one grid of one or more focusing elements disposed on an N-dimensional and arbitrarily shaped surface, at least one grid of one or more sensor elements disposed on an N-dimensional and arbitrarily shaped surface, and optionally, at least one grid of one or more stimulus guide elements disposed on an N-dimensional and arbitrarily shaped surface, where N can be chosen to be 1, 2, 3, or any other suitable quantity [50]. The grid of sensor ensembles illustrated here defer from the sets of equivalent methods, systems and devices described in the '274 patent in that the sensor ensembles described here are simpler and lack the focusing elements of the '274 patent. Furthermore, the present invention is further distinguished from the invention described in the '274 patent in the sense that the present invention requires the creation of a counterpart or corresponding environment for the biological substrate under consideration while the invention described in the '274 patent does not have any such requirement. Other distinguishing features are apparent from the details contained in this disclosure.

### Alternative Sensor Configurations

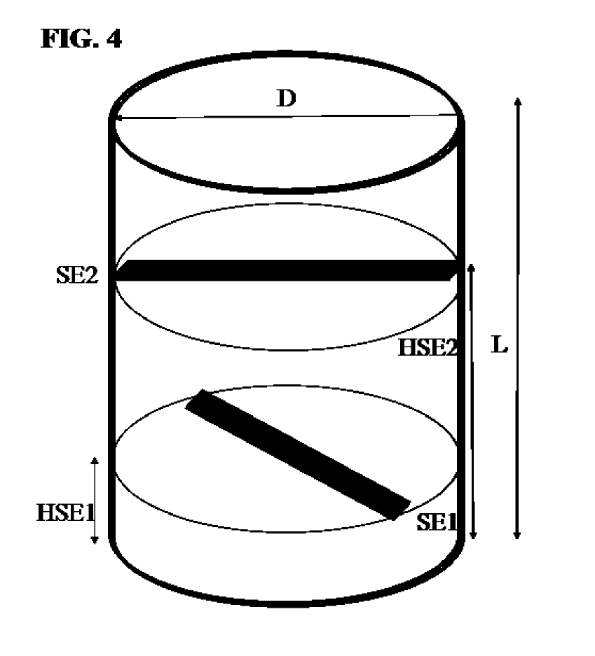

An alternative configuration in which two sensors could be placed orthogonally with a separation of 10.0 mm along the axis of the cylinder could be used to mitigate the putative effects of placing electrodes directly above each other as shown in FIG. 3. This alternative configuration is shown in FIG. 4 where the diameter of the containing plastic cylinder D could be 10.0 mm, its height L could be 20.0 mm, the height of the first electrode (SE1)—HSE1 could be 2.0 mm and the height of the second electrode (SE2)—HSE2 could be 12.0 mm—giving an inter-electrode distance of 10.0 mm.

Furthermore, consider the simple but effective sensor ensemble topography contained within a cylinder that partitions the cylinder into n (where n is 1 for one plane, 2 for two planes, 3 for three planes, and so on) unique planes passing through the axis of the cylinder with each plane separated by an angle of π/n radians from each of its nearest neighbors.

The edges of sensors or actuators (collectively transducers) in a configuration in which a single sensor or actuator or transducer is embedded in each plane of the cylinder—at a distinct height from the base—circumscribe a double helix.

One of ordinary skill in the art would appreciate that simple techniques such as the computation of the Pearson product-moment correlation coefficients, the display and inspection of scatter plots and the computation of the electrical distances between selected electrodes or sensors or actuators or transducers as well as any other suitable techniques would facilitate the comparison of the performance of this double helix topography with the referential arrangement in which sensors or actuators or transducers are stacked one above the other on a plane intersecting the axis of the cylinder.

### Cylinder Partitioning

The goal is to minimize the effects of inter-electrode interference in sensor or actuator or transducer ensembles comprising multiple electrodes or sensors or actuators or transducers. In the referential topography, sensors or electrodes or actuators or transducers could be stacked one above the other in the sensor or electrode or actuator or transducer ensemble.

Experimental results suggest that limiting overlap between neighboring sensors or electrodes or actuators or transducers could mitigate the effects of inter-electrode interference and lead to the acquisition (or generation) of more distinct signal streams with greater information content. Accordingly, the partitioning of the containing cylinder into unique planes passing through the axis of the cylinder is proposed. Sensors or electrodes or actuators or transducers can then be embedded in each unique plane at a distinct height from the base of the cylinder.

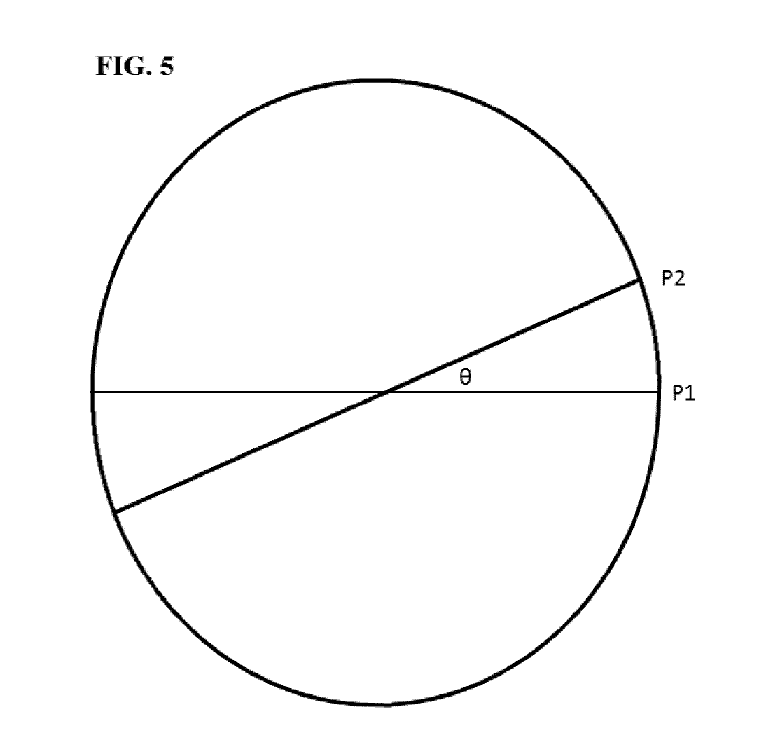

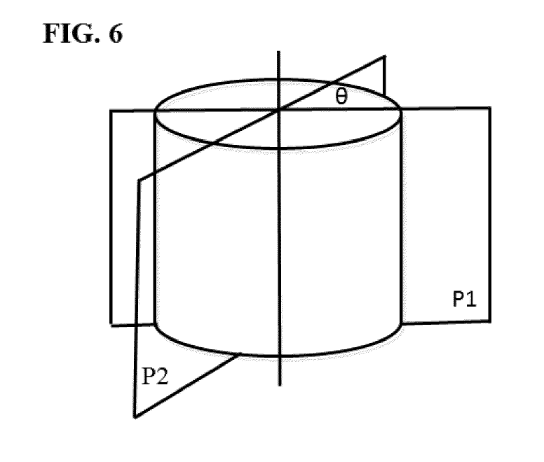

FIG. 5 is the top view of a containing cylinder outlining two representative planes P1 and P2. The angle between the planes is θ. In FIG. 6, the same arrangement is depicted as viewed from the side in three dimensions. The number of planes, n, is arbitrary in principle.

The following formula gives the inter-plane angle, θ, in terms of the number of planes, n.

$$θ = \frac{π}{n} ,$$

where θ is the inter-plane angle in radians and n is the number of planes.

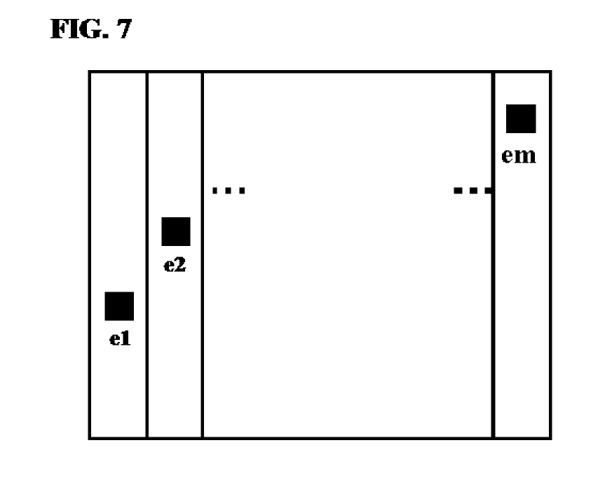

An arbitrary number of transducers, m, can be embedded within each unique plane. This can be done by subdividing the plane into m separate strips and embedding a transducer in each strip as shown in FIG. 7 where transducers e1, e2, . . . , em are embedded in the strips, one transducer per strip.

In practice, both the number of planes n (where n is 1 for one plane, 2 for two planes, 3 for three planes, and so on) and the number of strips per plane m (where m is 1 for one strip, 2 for two strips, 3 for three strips, and so on) are limited by the physical characteristics (principally the sizes) of the cylinder and transducers.

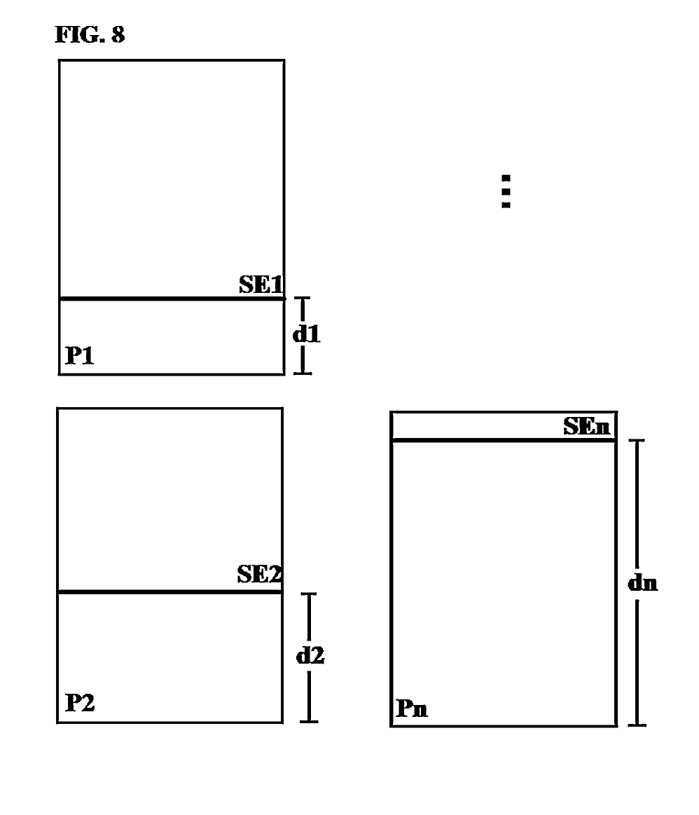

For simplicity, just one transducer could be embedded per plane with each transducer at a distinct height from the base of the cylinder. This arrangement is shown in FIG. 8 in which the planes are labeled P1, P1, . . . , Pn and the corresponding transducers are labeled SE1, SE2, . . . , SEn. Each transducer is located at a distinct height (depicted as d1, d2, . . . , dn) from the base of the cylinder. It is instructive to note that since each unique plane intersects the axis of the cylinder and contains a single transducer, the sensors overlap around the axis of the cylinder.

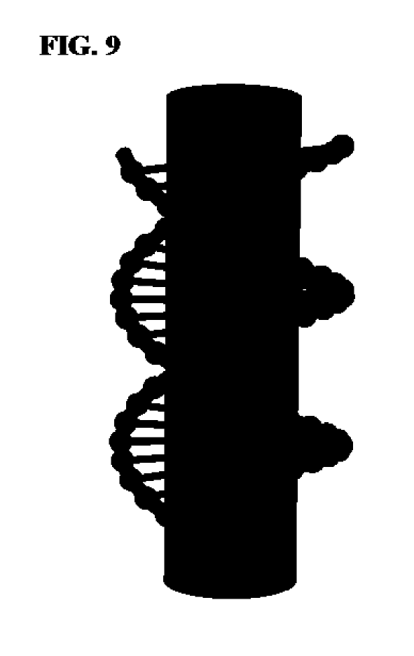

The regions of the transducers intersecting the surface of the cylinder in this simple configuration (one transducer per plane at a distinct height from the base of the cylinder) circumscribe a double helix as illustrated in FIG. 9.

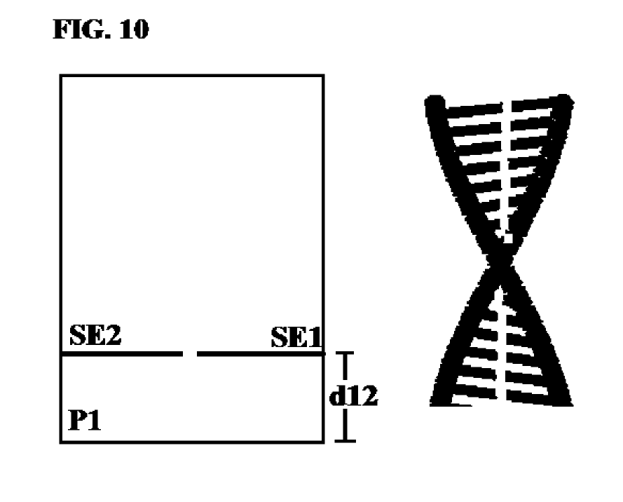

As noted previously, the double helical configuration with a single transducer per plane intersecting the axis of the cylinder leads to the overlap of transducers around the axis. This overlap can be removed by splitting each transducer into two transducers (one on each helix) with some space between them—resulting in the double-stranded helical topography depicted in FIG. 10.

FIG. 10 illustrates the splitting of a transducer on a plane P1 into two opposing transducers SE1 and SE2 at a height d12 from the base of the cylinder and the double-stranded double helix formed by partitioning the containing cylinder into unique planes and embedding two opposing and horizontally separated transducers per plane at a distinct height from the base of the cylinder.

### Configuration Data Analysis

What effect, if any, does the topography of the transducer ensemble have on the distinctness of the EEG or other data associated with a biological substrate recorded or generated by each of the transducers in the ensemble?

To investigate this question in the case where the transducers are configured as sensors or electrodes, the acquired EEG data could be analyzed using measures of the “electrical distance” between electrodes, the Pearson product-moment correlation coefficients between pairs of EEG signal streams corresponding to pairs of electrodes and scatter plots for electrode pairs while being cognizant of the actual spatial distances between the electrodes.

### Computing Correlation Coefficients

For any pair of electrodes, the Pearson product-moment correlation coefficient (r) can be calculated thus:

$$r = \frac{{\sum }_{i = 1}^{n} ( {X}_{i} - \overline{X} ) ( {Y}_{i} - \overline{Y} )}{\sqrt{{\sum }_{i = 1}^{n} {( {X}_{i} - \overline{X} )}^{2}} \sqrt{{\sum }_{i = 1}^{n} {( {Y}_{i} - \overline{Y} )}^{2}}}$$

where X̅ denotes the sample mean for EEG or any other suitable type of data recorded at the first electrode, Y̅ is the sample mean for EEG or any other suitable type of data recorded at the second electrode, and n is the number of samples.

### Measuring Electrical Distance Using the Hjoth Laplacian

A linear approximation to the surface Laplacian can be computed using the Hjorth algorithm [51]. In the Hjorth waveform Hᵢ (t, N), the contribution to the signal from each electrode i is expressed as the difference between the time-varying potential Pᵢ (t) and the scaled sum of the potentials Pⱼ (t) at each of N neighboring electrodes, as expressed in the following equation:

$${H}_{i} ( t , N ) = {P}_{i} ( t ) - {\sum }_{j = 1}^{N} {P}_{j} ( t ) {W}_{i - j} ( N ) ,$$

where Wᵢ₋ⱼ is a weighting factor for each neighbor that is inversely proportional to the distance dᵢ₋ⱼ between the electrodes.

$${W}_{i - j} ( N ) = \frac{\frac{1}{{d}_{i - j}}}{{\sum }_{k = 1}^{N} \frac{1}{{d}_{i - k}}}$$

In the intrinsic Hjorth algorithm, the spatial distance dᵢ₋ⱼ is replaced by the non-spatial “electrical distance” Dᵢ₋ⱼ reflecting the electrical similarity between electrodes i and j. The potential difference waveform Pᵢ₋ⱼ(t) between two electrodes i and j is given by:

Pᵢ₋ⱼ(t)=Pᵢ(t)−Pⱼ(t)

The “electrical distance” can then be computed as:

$${D}_{i - j} = \frac{1}{T} {\sum }_{t = 1}^{T} {( {P}_{i - j} ( t ) - \overline{{P}_{i - j} ( t )} )}^{2}$$

In foregoing equation for the computation of the “electrical distance”, P̅ᵢ̅₋̅ⱼ̅(̅t̅)̅ is the mean potential difference waveform. This gives the temporal variance of the difference potential waveform.

According to [52], replacing the spatial distance dᵢ₋ⱼ with the temporal variance of the difference potential waveform Dᵢ₋ⱼ yields the intrinsic Hjorth transform in the case of a single neighbor. In order to detect electrolyte bridges between electrodes, it is sufficient to limit consideration of electrical distances to the detection of the single nearest neighbor. This is equivalent to setting N=1 in the equation describing the linear approximation to the surface Laplacian that can be computed using the Hjorth algorithm [52].

### Discussion

The apparent presence of electrolyte bridges in the sensor ensembles should preclude the measurement of distinct signal streams as explained in references [53, 54] and should yield correlation coefficients close to 1.0. As already noted, contemporary EEG systems typically acquire a single signal stream per site and are configured in a manner that virtually precludes the simultaneous acquisition of multiple signal streams per site. Furthermore, since the tangential separation of the electrodes in the configurations presented in proposed experimental setup described in this disclosure is much smaller than 10.0 mm, tangential contributions should be substantially identical (see references [39-44]) with a correlation coefficient of 1.0. However, experimental results illustrate that distinct signal streams can indeed be obtained from sensors placed at the same site on the surface representing the source of the bio-signals. The signals acquired are likely to originate from sources located at different depths (in a radial direction) and reach the sensors at different times due to propagation delays in the intervening media. The results seem to indicate that the correlation between signals tends to decrease as the inter-electrode distance increases in the radial direction. Placing the electrodes orthogonally as shown in FIG. 4 could result in a significant decrease in the correlation coefficient and could make it possible to notice differences between the signals via mere visual inspection—suggesting that this could improve the amount of additional independent information that can be acquired at the same site using this arrangement.

The embodiments enabled by the present invention provide means of significantly increasing the spatial resolution of bio-signal acquisition and generation systems with the potential to provide new insights into the underlying physiological processes and expand the range of applications of these systems. New insights gained from the additional information acquired or generated using systems based on the concepts presented here could potentially open up new avenues in both clinical and non-clinical applications of bio-signals. Systems, methods, devices and experiments for the demonstration and application of simultaneous non-invasive acquisition or generation of distinct bio-signals at different layers or depths within the bio-signal source at a single transducer site have been disclosed.

Using invasive cortical implants, researchers obtained direct evidence for acoustic-to-higher order phonetic level encoding of speech sounds in human language receptive cortex [55]. However, similar or better results—including the precise specification of which layer within the cortex is implicated in which aspect of speech sound processing—could be obtained non-invasively using bio-signal acquisition or generation systems based on the principles of the present invention.

The application of the results enabled by the principles of the present invention would provide new insights into the functional organization of the brain and other biological systems in which signals at different layers contribute to system behavior. It should therefore be obvious to one of ordinary skill in the art that the principles of the present invention enable a very wide range of clinical and non-clinical application domains.

### Multilayer EEG Color or Feature Maps

The capabilities of systems based on the principles of the present invention could be harnessed to create multi-layer data navigation applications where the data is associated with stimuli acquired or generated at multiple layers within a biological substrate.

One such application is a multi-layer color map capable of translating sensor values into corresponding colors in which each layer within the color map corresponds to a layer within the underlying biological substrate. This can be used to navigate and inspect the data from the biological substrate in a manner that enables new relationships to be uncovered and existing relationships to be better characterized and understood. Such applications can be used to diagnose diseases that are hitherto impossible or difficult to diagnose with the prior art. Furthermore, better understanding of the underlying biological substrate can be acquired.

Note that the data associated with any transducer or corresponding biological substrate site could be transformed or characterized and presented as features in any suitable manner based on the demands of a specific application. For example, stimuli strengths at specific transducer sites could be transformed into pixel values in an image representing the transducers. In this case the exact nature of the pixel values could be adapted to specific applications. For example, the pixel values could be interpreted as representing suitably formatted colors as appropriate. Any other interpretation suitable to any specific application is also acceptable. Other representations include the use of bars—as in a bar code scenario—with heights that correspond to the strengths or other selected aspect of the associated transducers or corresponding biological substrate site.

Efficient data management techniques pertaining to such applications including the use of predictive loading of relevant data and possible subsequent presentation on a display window or computer monitor or any other suitable device or system based on a dynamic prediction of the user's point of view within the data stream could be applied to enable practical implementation and acceptable performance of this multi-layer color map and related applications on off-the-shelf personal computer systems.

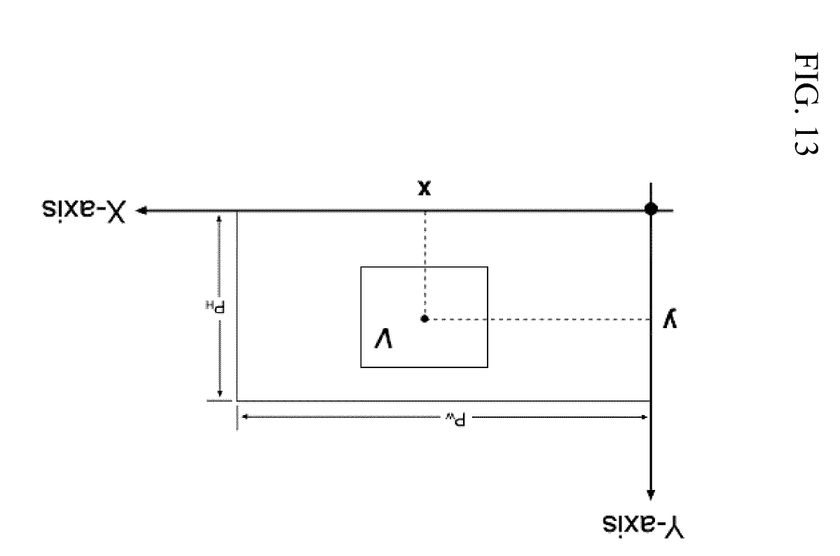

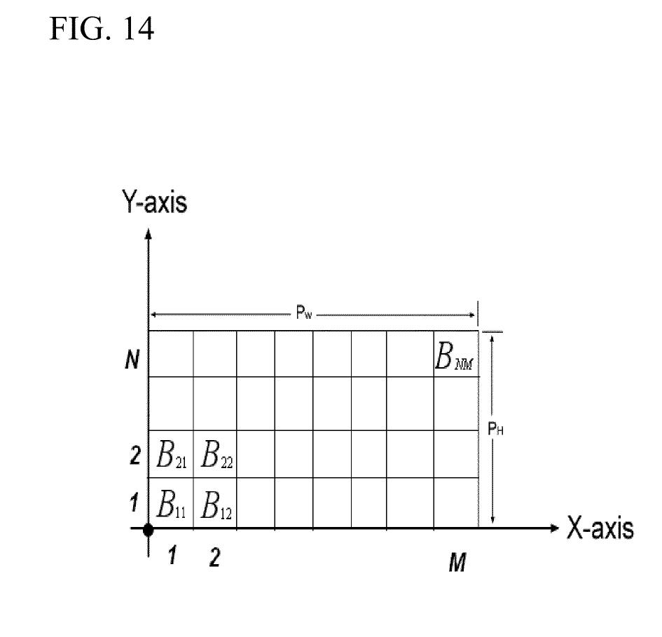

When data associated with selected transducer or corresponding biological substrate site is characterized in the form of pixel values of images representing the data, dynamic view prediction of the user's field of view and associated intelligent data management techniques could be used to facilitate efficient data navigation. Based on the observation that interactive rendering of the image data set involves the display of a relatively small (compared to the size of the underlying image data itself) view window, the preferred embodiment of the present invention teaches the use of a robust two-tier or bi-level representation of the image. The first level contains a virtual view of an entire image frame as a single continuous set of pixels. FIG. 13 illustrates the first level for a two-dimensional image frame of width P_{W} and height P_{H} pixels. The region of interest or view window is indicated as V in FIG. 13. Since a single image frame can be very large, it is generally impractical to attempt to load the entire image frame (typically corresponding to tens of gigabytes of physical memory) into memory at once. Consequently, the second level comprises a segmentation or partitioning of each image frame into distinct image blocks of a size and color depth that facilitates straightforward manipulation on an average personal computer. This partitioning scheme is shown in FIG. 14, where the image of FIG. 13 has been segmented into N×M distinct image blocks labeled B₁₁, B₁₂, . . . , B_{NM}. The size of each partition can be chosen such that the view window, V, straddles just a couple of image blocks. In this case only those image blocks in the second layer that are covered or straddled by the view window need be loaded into memory for the manipulation or rendering of the view, leading to a significantly reduced memory footprint.

The use of a two-tier image representation scheme permits alternate views of the image data that make further manipulation easier. For example, the simplicity of the first level permits the applications of a multi-resolution pyramid representation of the image data, such as that described by Peter J. Burt et al. in “The Laplacian Pyramid as a Compact Image Code”, IEEE Transactions on Communications, 1983, pp. 532-540, for efficient compression, storage and transmission and optionally for adaptive rendering that maintains a constant frame rate. A thumbnail of the entire image could also be generated at the first level. Such a thumbnail could be used to display a lower resolution version of the view window while waiting for image data to be retrieved and/or decompressed. Furthermore, the dynamic view prediction and on-demand loading algorithms described hereinafter are readily applicable to the second tier's image block representation.

Following is an outline of the process of visualizing the data sets according to the preferred embodiment. First, a view window V is specified as illustrated in FIG. 13. The view window represents the segment of the current image frame that is indicated by the view parameters. In a given implementation, three view parameters such as the pan angle, the tilt angle or azimuth and the zoom or scale factor could be used to control the view. Other relevant view parameters or factors could be considered as appropriate for any given application. User input could be received via the keyboard and/or mouse clicks within the view window. Suitable gesture recognition interfaces or touch-based interfaces or brain-computer interfaces or any other suitable interface could be used to receive input, provide feedback or generally enable user interaction. Alternatively, a head-mounted display and orientation sensing mechanism could also be used. Views could be generated based on view window size and received input. In order to facilitate interactive rendering without the latencies and other limitations associated with the prior art, the rate of change of each of the view parameters with respect to time could be computed dynamically. The computed rate of change could then be used to predict the value of the parameter at any desired time in the past or future.

The following equation illustrates the use of the dynamic view prediction algorithm for a specific view parameter P—which could be the pan, tilt, zoom level, or any other suitable parameter.

p=p₀+KaT

In the foregoing equation, P is the predicted value of the parameter at time T, p₀ is the current value of the parameter, a is the dynamically computed rate of change of the parameter with respect to time and K is a scale factor, usually 1. The values of the parameters predicted by the foregoing equation could be used to determine which specific image blocks need to be loaded into memory at any given time.

A computer software implementation using a background thread dedicated to loading those image blocks that are covered by the current view as well as any additional image blocks that might be needed for rendering the view in the future or past, that is, a number of future or past time steps, could be used. Since the number of image blocks per frame is usually small, it is practical to preload, pre-fetch or pre-synthesize as appropriate, image blocks that would be required for rendering several time steps ahead or behind—permitting smooth rendering at real-time rates. The image data could be distributed from a server over the Internet or other network or accessed from local storage on a host computer. Any other alternative source and method of distribution could be used where appropriate.

Studies with image visualization systems have consistently shown that the use of a damping or inertial function to facilitate gradual changes in view parameters leads to the perception by users of a vastly smoother, more natural and more intuitive viewing process. This observation can be exploited by the dynamic view prediction algorithm of the present invention to provide smooth, interactive distribution and visualization of very large image data sets even over relatively slow network connections such as the current Internet and other bandwidth-limited scenarios without the latencies and other deficiencies associated with the prior art. The gradually changing view parameters would then permit many more future or past time steps to be computed, preloaded, pre-fetched and/or pre-synthesized as appropriate to a greater degree of accuracy.

It should be noted that while the foregoing preferred embodiments for the management of very large data sets from systems based on the principles of the present could be appropriate in situations where memory, computing and associated resources are limited with respect to the amounts of data processing required for effective utilization of the system, much simpler and more straightforward data management techniques could be employed in situations with fewer data points from the transducers or other system components without deviating from the present invention.

A wide variety of systems and schemes could be adopted in the representation and/or presentation of the data from the sensors. For example, multiple windows on a suitable graphical computer operating system or multiple monitors or display devices could be utilized in displaying the data with the image or representation for a specific layer within the biological substrate assigned to a specific window or computer monitor or display device. Alternative modalities for the representation and/or presentation of the data from the sensors include the use of a mouse or joystick or gesture recognition interface or touch-based interface or brain-computer interface or any other suitable interface to allow the user to navigate the data by stepping through the data—revealing information from different layers within the biological substrate in the process. In this alternative scenario, a single window or a single display device or computer monitor could be employed in displaying only the data associated with the layer within the biological substrate that the user is currently interested in viewing or interacting with. One of ordinary skill in the art would appreciate that numerous alternative means and methods could be utilized in the representation and/or presentation of the data from the sensors.

In such multi-layer display applications, resolution could be improved by utilizing techniques such as tri-linear interpolation to compute data for positions without sensors in the acquisition system by applying the data from neighboring sensor sites.

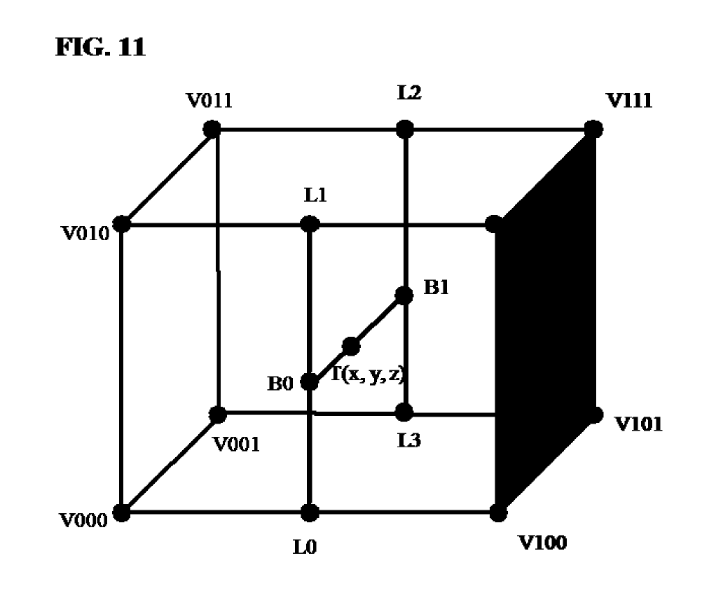

FIG. 11 depicts tri-linear interpolation for a site bounded by eight neighboring transducer sites in a 3-dimensional spatial configuration and illustrates how tri-linear interpolation could be implemented. For example, to compute data for the point labeled T(x, y, z) located at 3D coordinates (x, y, z) from an arbitrarily chosen origin and for which sensor data is not directly available, sensor data for the neighboring sensor sites labeled V000, V100, V101, V001, V010, V110, V111 and V011 could be utilized as follows and tri-linear interpolation applied.

T(x,y,z)=(V00*(1−x)+V100*x)*(1−y)+((V010*(1−x)+V110*x)*y)*(1−z)+V001*(1−x)+V101*x)*(1−y)+((V011*(1−x)+V111*x)*y)*z

Note that L0, L1, L3, L2 and B0, B1 are intersection points between adjacent faces of the cube formed by the neighboring sensor sites (namely V000, V100, V101, V001, V010, V110, V111 and V011) for which data is available and the target site—T(x, y, z)—for which no sensor data is available.

It should be obvious to one of ordinary skill in the art that although the foregoing multi-layer data navigation applications based on the principles of the present invention involve color mapping or bar code or similar presentation or feature mapping, any other suitable transformation or representation of the data or any interpretation or characterization of the data into salient or relevant features or feature maps could be employed in implementing a multi-layer data navigation application.

Furthermore, it should be obvious to one of ordinary skill in the art that the foregoing multi-layer data navigation applications based on the principles of the present invention could be utilized in reverse to permit the modulation or moderation or control or stimulation of a biological substrate via interaction with or manipulation of the characterization of the data from the sensors or for the actuators. For example, an interface could be provided that allows the viewer of the data to interactively modify any desired aspects of the presented data such as the heights of bars or suitable color representations corresponding to the strength of stimuli at specific transducer (sensor or actuator) sites within the counterpart environment and thus cause the associated actuator or transducer to emit or transmit or moderate or modulate stimuli that influence associated aspects of the biological substrate in any desired manner Many other uses of the capability for multi-layer data navigation are possible.

### Improving the Information Transfer Rate of Brain-Computer Interfaces

As noted earlier, the spatial resolution of contemporary EEG systems is quite low—with systems typically comprising between 1 and 256 electrodes, each of which aggregates signals from massive neuronal populations. By utilizing the systems enabled by the principles of the present invention, the spatial resolution of EEG systems can be significantly increased.

Signals acquired using systems implemented in accordance with the principles of the present invention could be further processed to facilitate brain-computer interfaces (BCIs), medical diagnosis or research on the bio-signals. As one of ordinary skill in the art would readily notice, the signals could also be transmitted using wires, wirelessly, optically or via any other suitable means to any device or system adapted to further process the signals. Such a device or system could be dedicated hardware implemented using field-programmable gate arrays (FPGAs), application-specific integrated circuits (ASICs) or general-purpose computers executing suitable computer-readable instructions embodying the methods used in the processing. The signals could also be visualized using suitable hardware and/or software systems to facilitate navigation and study of the signals.

In the case of EEG signals, neuronal populations located at varying depths within the brain contribute to the signals detected on the scalp. With contemporary EEG systems typically containing a single electrode or sensor per site, it is impractical to extract signals from specific layers or regions of the brain but only a single signal (presumably a combination of signals) from a given neuronal population. By placing a plurality of sensors within an ensemble of sensors for a given site or neuronal population, the present invention provides a means for the extraction of signals from varying depths within the site. If all sensors within an ensemble are sampled substantially simultaneously, then the signal extracted from each sensor within the ensemble could be adapted to represent the contribution of a subset or sub-layer of the target neuronal population. Although in practice the signal extracted from a given sensor within the ensemble might be a superposition of signals from multiple layers or subsets of neurons within the target neuronal population, it is feasible to isolate signals from specific sub-layers or subsets of neurons possibly by taking the configuration of the individual sensors into account. Isolation of signals from specific sub-layers or subsets of neurons could also be feasible by sampling the sensors in a predetermined pattern. The sensors or electrodes need not be sampled simultaneously in order to isolate signals from specific depths, layers or populations. The topology or placement of sensors within the ensemble could also be designed to facilitate the isolation of contributions from specific sub-layers or subsets of neurons.

Although the foregoing description focuses on the “passive” use of the sensor ensembles for the acquisition of bio-signals, the ensembles could be used in reverse to excite targeted neuronal populations and/or to transmit low-power pulses into the signal source and then acquire and analyze the resulting interference to locate and/or otherwise characterize the source of the interference. This would constitute an “active” use of the sensor ensembles.

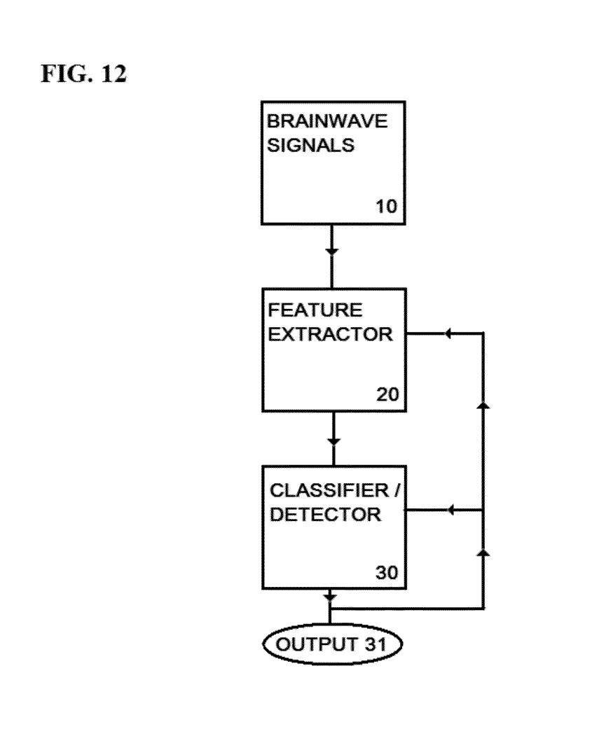

In FIG. 12, brainwave signals corresponding to a subject's physiological or event-related cognitive state are acquired by the brainwave acquisition unit, 10. A suitable recording device based on electroencephalograph, electrocorticograph, near-infrared spectrograph, and so on, could be used as the source of the brainwave signals from the subject. The spatial and temporal resolution of contemporary brainwave recording equipment is limited. Using an ultra-dense sensor network (possibly comprising nano-probes/nano-electrodes) capable of recording the activity (electrical, electromagnetic, and so on) of individual neurons or neural populations consisting of a relatively small number of neurons (in the order of 1 to 100 neurons per population), more accurate brainwave readings could be obtained.

Numerous studies (please refer to the appended list of references for examples) have shown that it is valid to consider information processing in human (and other animal) brains as a hierarchical and distributed model in which information representing stimuli or physiological states could be decomposed into simpler units of information and the processing of these simpler units distributed among different neural populations. The present invention permits the adoption of this approach to the processing of brainwave signals. Accordingly, the feature extraction unit—depicted generally as 20 in FIG. 12—extracts representations of salient features from the incoming brainwave signals. The exact features selected and how these are represented depends on the application. For a given classification task, a set of salient features is selected by a separate feature extraction unit. Each feature extraction unit is coupled to a classification/detection unit, 30, that is trained to recognize/detect that specific feature. The classification units preferably classify/detect features in parallel. With the decreasing cost of multi-core computers and refinements in parallel programming languages and systems, this scheme could be amenable to straightforward implementation on general-purpose consumer personal computers. In the absence of multi-core hardware, multi-threaded programming could be used to implement parallel feature processing. The output of the classifier/detector, labeled 31 in FIG. 12, is fed back to the feature extractor, 20 and classifier, 30 and used to adaptively modify the behavior of the feature selector and/or classifier with a view to providing more accurate feature selection and/or classification. This processing is repeated (preferably in parallel) for each feature at each stage of the hierarchy with the classification results from all salient features for each target class recombined to generate the final output which in turn could be used to control external devices. Jonathan R. Wolpaw and Dennis J. McFarland describe an adaptive algorithm that uses a simple linear combination of relevant features to improve the effectiveness of a non-invasive BCI designed for 2-dimensional computer cursor control in United States National Academy of Sciences (2004 Dec. 21; 101(51): 17849-17854). The method described by Wolpaw et al. is limited by the requirement for extensive training of the user. In contrast, the present invention could be directed as described towards a method that uses the hierarchical decomposition of the feature space to provide a means of identifying and adaptively modifying/classifying simpler features (that are more likely to have characteristics common to most subjects) in parallel which are then re-combined to generate the final output thus obviating or at least mitigating the need for extensive subject training. This increases the information transfer rate (simpler features can be classified faster and more accurately in parallel using simpler algorithms) and expands the scope of practical applications of BCIs.

The output of the classifier/detector, labeled 31 in FIG. 12, could be utilized to control external systems or devices such as the position of a 2-dimensional cursor on a computer screen or to trigger events in a suitably configured computer application or to perform any desired task in any suitably configured or adapted system. Results from the output of the classifier/detector could also be applied to the extraction of usable information such as letters, words, images, concepts, emotional states or any other useful information from the subject or biological substrate.

It should be noted that for applications that do not require justifiably stringent performance levels from the signal processing system, the output of the classifier/detector, labeled 31 in FIG. 12, could be utilized directly, for example by being fed directly into any suitably configured or adapted downstream application or system (such as for the control of the position of a 2-dimensional cursor on a computer screen or to trigger events in a suitably configured computer application or to perform any desired task in any suitably configured or adapted system) without recourse to the feedback loop. Thus the step of feeding the results of the classifier/detector back to the feature extractor or back into any other part of the signal processing system could be omitted as required by any specific application.

Furthermore, it should be understood that for numerous applications, a single signal processing pipeline could be sufficient. Thus the use of parallel signal processing streams as described in the foregoing disclosure could be avoided as required by any specific application in favor of the simpler single signal processing stream running from the acquisition of the requisite brainwave signals (labeled 10 in FIG. 12) down to the output of the classifier/detector, labeled 31 in FIG. 12

### Information Management

Any information associated with any aspect of any embodiment of the present invention could be managed as elements in a universal file format. Such a universal file format would specify a header identifying the file type and containing information as to the number, types, locations and sizes of the elements it contains. Each element in the file is in turn described by a header specifying the type of the element, its size and any relevant data or attributes and the types, locations and sizes of any additional elements it contains. By making use of self-describing elements in the manner explained in the foregoing, the universal file format would be able to store an arbitrary element having an arbitrary number and types of other such elements embedded in it. For a more concrete and specific example, information associated with data for visualization of states of a biological substrate could be managed as elements in the universal file format described in the foregoing. Furthermore, information associated with any transformations required to translate any aspects of a biological substrate such as a characterization of its state in a suitable mathematical or other form into commands for the control of external systems such as the position of a cursor on a computer system display could also be managed as elements in the universal file format described in the foregoing.

### Alternative Embodiments

The foregoing description of the preferred embodiments of the present invention disclosed specific systems, devices, algorithms, experimental setups, mathematical analyses, stimulus types, and so on. In particular, systems based on electrical or electrochemical stimuli were disclosed. However, one of ordinarily skill in the art would readily appreciate that any other suitable types of stimuli including chemical, electrical, magnetic, optical, acoustic, mechanical, electromagnetic, ultrasound, microwave, radio, gamma ray, x-ray, ultraviolet light, white light, infrared light, laser, or any other stimuli associated with biological substrates—both living and non-living or both animate and inanimate—could be utilized in implementing the present invention.

It should be understood that numerous alternative embodiments and equivalents of the invention described herein may be employed in practicing the invention and that such alternative embodiments and equivalents fall within the scope of the present invention.

### NON-PATENT REFERENCES (PATENT REFERENCES ARE EMBEDDED IN THE RELEVANT SECTIONS OF THE SPECIFICATION)

[1] Ian T. Baldwin and Jack C. Schultz, “Rapid Changes in Tree Leaf Chemistry Induced by Damage: Evidence for Communication Between Plants”, Science, Vol. 221, no. 4607, pp. 277-279, (1983).
[2] R. Karban, I. T. Baldwin, K. J. Baxter, G. Laue and G. W. Felton, “Communication Between Plants: induced resistance in wild tobacco plants following clipping of neighboring sagebrush”, Oecologia, 125, pp. 66-71, (Springer-Verlag 2000).
[3] J. D. Pye, “A theory of echolocation by bats”, Journal of Laryngology and Otology, 74, pp. 718-729, 1960.
[4] J. D. Pye, “Perception of distance in animal echolocation”, Nature, Lond., 190, pp. 362-363, (1961).
[5] J. D. Pye, M. Flinn and A. Pye, “Correlated orientation sounds and ear movements of horseshoe bats”, Nature, Lond., 196, pp. 1185-1188, (1963).
[6] C. F. Moss and M. Zagaeski, “Acoustic information available to bats using frequency modulated sounds for the perception of prey”, J. Acoust. Soc. Am. 95, 2745-2756, (1994).
[7] M. E. Jensen, L. A. Miller and J. Rydell, “Detection of Prey in s Cluttered Environment by the Northern Bat Eptesicus Nilssonii”, Journal of Experimental Biology, 204, pp. 199-208, (2001).
[8] R. W. Murray, “The Response of Ampullae of Lorenzini of Elasmobranchs to Electrical Stimulation,” Journal of Experimental Biology,”, 39, pp. 119-128, (1962).
[9] R. W. Murray, “Electrical Sensitivity of the Ampullae of Lorenzini”, Nature, 187, p. 957, (1960).
[10] A. J. Kalmijn, “Electro-perception in Sharks and Rays”, Nature, 212, pp. 1232-1233, (1966).
[11] A. J. Kalmijn, “Electric and magnetic field detection in elasmobranch fishes”, Science 26, Vol. 218, no. 4575, pp. 916-918, (1982).
[12] B. R. Brown, “Modeling an electro-sensory landscape: behavioral and morphological optimization in elasmobranch prey capture”, Journal of Experimental Biology, 205, pp. 999-1007, (2002).
[13] M. W. Jenkins, A. R. Duke, S. Gul, Y. Doughman, H. J. Chiel, H. Fujioka, M. Watanabe, E. D. Jansen and A. M. Rollins, “Optical pacing of the embryonic heart”, Nature Photonics, 4, pp. 623-626, (2010).
[14] S. Furnan, J. B. Schwedel, “An Intracardiac Pacemaker for Stokes-Adams Seizures”, Pacing and Clinical Electrophysiology, Vol. 29, Issue 5, pp. 453-458, (2006).
[15] A. Bohm, A. Pinter, A. Szekely, and I. Preda, “Clinical Observations with Long-term Atrial Pacing”, Pacing and Clinical Electrophysiology, Vol. 21, Issue 1, pp. 246-249, (1998).
[16] D. Halperin, T. S. Heydt-Benjamin, B. Ransford, S. S. Clark, B. Defend, W. Morgan, K. Fu, T. Kohno, W. H. Maisel, “Pacemakers and Implantable Cardiac Defibrillators: Software Radio Attacks and Zero-Power Defenses”, IEEE Symposium on Security and Privacy, pp. 129-142, (2008).
[17] A. Yatani, K. Okabe, J. Codina, L. Birnbaumer, A. M. Brown, “Heart rate regulation by G proteins acting on the cardiac pacemaker channel”, Science, Vol. 249, no. 4973, pp. 1163-1166, (1990).
[18] T. Hoshi, W. N. Zagotta, R. W. Aldrich, “Biophysical and molecular mechanisms of shaker potassium channel inactivation.”, Science 250, pp. 533-538, (1990).
[19] A. L. Hodgkin, A. F. Huxley, “The components of membrane conductance in the giant axon of Loligo.”, J. Physiol. (Lond) 116 (4), pp. 473-496, (1952).
[20] A. L. Hodgkin, A. F. Huxley, “Currents carried by sodium and potassium ions through the membrane of the giant axon of Loligo.”, J. Physiol. (Lond.) 116(4), pp. 449-472, (1952).
[21] A. L. Hodgkin, A. F. Huxley, “The dual effect of membrane potential on sodium conductance in the giant axon of Loligo.”, J. Physiol. (Lond.) 116(4), pp. 497-506, (1952).
[22] A. L. Hodgkin, A. F. Huxley, “A quantitative description of membrane current and its application to conduction and excitation in nerve.”, J. Physiol. (Lond.) 117(4), pp. 500-544, (1952).
[23] A. L. Hodgkin, A. F. Huxley, B. Katz, “Measurement of current-voltage relations in the membrane of the giant axon of Loligo.”, J. Physiol. (Lond.) 116(4), pp. 424-448, (1952).
[24] D. Noble, “Application of Hodgkin-Huxley equations to excitable tissues.”, Physiol. Rev. 46(1), pp. 1-50, (1966).
[25] E. Neher, B. Sakmann, B. Katz, “Single-channel currents recorded from membrane of denervated frog muscle fibers.”, Nature 260, pp. 799-802, (1976).
[26] E. Neher, B. Sakmann, B. Katz, The patch clamp technique.”, Sci. Am. 266:(3), pp. 28-35, (1992).
[27] 0. P. Hamill, A. Marty, E. Neher, B. Sakmann, F. J. Sigworth, “Improved patch clamp techniques for high resolution current recording from cells and cell-free membranes.”, Pflüger Arch. ges. Physiol. 391, pp. 85-100, (1981).
[28] B. Sakmann, E. Neher, “Patch clamp techniques for studying ionic channels in excitable membrane.”, Annu. Rev. Physiol. 46, pp. 455-472, (1984).
[29] K. S. Cole, J. W. Moore, “Potassium ion current in the squid giant axon: Dynamic characteristics.”, Biophys. J. 1(1): pp. 1-14, (1960).
[30] C. W. Armstrong, B. Hille, “The inner quaternary ammonium ion receptor in potassium channels of the node of Ranvier.”, J. Gen. Physiol. 59: pp. 388-400, (1972).
[31] E. Neher, A. Marty, “Discrete changes of cell membrane capacitance observed under conditions of enhanced secretion in bovine adrenal chromaffin cells.”, Proc. Nat. Acad. Sci. USA 79: pp. 6712-6716, (1982).
[32] P. N. T. Unwin, G. Zampighi, “Structure of the junctions between communicating cells.”, Nature 283: pp. 545-549, (1980).
[33] C. Toyoshima, N. Unwin, “Ion channel of acetylcholine receptor reconstructed from images of postsynaptic membranes.”, Nature 336: pp. 247-250, (1988).
[34] J. B. Patlak, M. Ortiz, “Two modes of gating during late Na+ channel currents in frog sartorius muscle.”, J. Gen. Physiol. 87: pp. 305-326, (1986).
[35] M. E. Phelps, J. C. Mazziotta, “Positron emission tomography: human brain function and biochemistry.”, Science Vol. 228, no. 4701: pp. 799-809, (1985).
[36] D. Cohen, E. A. Edelsack, J. E. Zimmerman, “Magnetocardiograms taken inside a shielded room with a superconducting point contact magnetometer.”, Applied Physics Letters 16(7): pp. 278-280, (1970).
[37] D. Cohen, “Magnetoencephalography: detection of the brain's electrical activity with a superconducting magnetometer.”, Science. 175: pp. 664-666, (1972).
[38] J. E. Zimmerman, P. Theine, J. T. Harding, “Design and operation of stable rf-biased superconducting point-contact quantum devices, etc.”, Applied Physics Letters 41: pp. 1572-1580, (1970).
[39] J. Malmivuo, V. Suihko, and H. Eskola, “Sensitivity distributions of EEG and MEG measurements.”, IEEE Trans. Biomed. Eng., vol. 44, pp. 196-208, (1997).
[40] T. F. Oostendorp, J. Delbecke, and D. F. Stegman, “The conductivity of the human skull: Results in vivo and in vitro measurements.”, IEEETrans. Biomed. Eng., vol. 47, pp. 1487-1492, (2000).
[41] S. Rush and D. A. Driscoll, “EEG-electrode sensitivity—An application of reciprocity.”, IEEE Trans. Biomed. Eng., vol. 16, Issue 1, pp. 15-22, (1969).
[42] M Junhofer, T Elbert, D. M. Tucker and B. Rockstroth, “Statistical control of artifacts in dense array EEG/MEG studies.”, Psychophysiology., 37, pp. 523-532, (2000).
[43] W. J. Freeman, M. D. Holmes, B. C. Burke, S. Vanhatalo, “Spatial spectra of scalp EEG and EMG from awake humans.”, Clin. Neurophysiol., Vol. 114, Issue 6, pp. 1053-1068, (2003).
[44] O. Vä isänen and J. Malmivuo, “Improving the SNR of EEG generated by deep sources with weighted multielectrode leads.”, Journal of Physiology-Paris., vol. 103, Issue 6, pp. 306-314, (2009).
[45] D. Cohen, B. N. Cuffin, K. Yunokuchi, R. Maniewski, C. Purcell, G. R. Cosgrove, J. Ives, J. G. Kennedy, and D. L. Schomer, “MEG versus EEG localization test using implanted sources in the human brain.”, Annals of Neurology., 28, Issue 6, pp. 811-817, (1990.
[46] H. Hallez, B. Vanrumste, R. Grech, J. Muscat, W. D. Clercq, A. Vergult, Y. D'Asseler, K. P. Camilleri, S. G. Fabri, S. V. Huffel and I. Lemahieu, “Review on solving the forward problem in EEG source analysis.”, Journal of NeuroEngineering and Rehabilitation., 4:46 doi:10.1186/1743-0003-4-46. (2007).
[47] M. Junghöfer, T. Elbert, P. Leiderer, P. Berg, B. Rockstroh, “Mapping EEG-potentials on the surface of the brain: a strategy for uncovering cortical sources.”, Brain Topogr. 9(3), pp. 203-217. (1997).
[48] J. R. Wolpaw and D. J. McFarland, “Control of a two-dimensional movement signal by a noninvasive brain-computer interface in humans.”, Proc Natl Acad Sci USA. 101 (51), pp. 17849-17854. (2004).
[49] W. J Freeman, “Use of Spatial Deconvolution to Compensate for Distortions of EEG by Volume Conduction”, IEEE Transactions on Biomedical Engineering, Vol. BME-27, Issue 8, pp. 421-429. (1980).
[50] F. E. Ekpar, “Method and Apparatus for Creating Interactive Virtual Tours”, U.S. Pat. No. 7,567,274, (2009).
[51] B. Hjorth, “Source derivation simplifies topographical EEG interpretation, Am J EEG Technol 20: pp. 121-132 (1980). American Journal Of Eeg Technology (1980)
[52] C. E. Tenke, J. Kayser, “A convenient method for detecting electrolyte bridges in multichannel electroencephalogram and event-related potential recordings.”, Clinical Neurophysiology 112, pp. 545-550. (2001).
[53] L. L. Greischar, C. A. Burghy, C. M. van Reekum, D.C. Jackson, D. A. Pizzagalli, C. Mueller, R. J. Davidson, “Effects of electrode density and electrolyte spreading in dense array electroencephalographic recording.”, Clinical Neurophysiology 115, pp. 710-720. (2004).
[54] C. E. Tenke, J. Kayser, “A convenient method for detecting electrolyte bridges in multichannel electroencephalogram and event-related potential recordings.”, Clinical Neurophysiology 112, pp. 545-550. (2001).
[55] E. F. Chang, J. W. Rieger, K. Johnson, M. S. Berger, N. M. Barbaro, R. T. Knight, Categorical speech representation in human superior temporal gyrus. Nature Neuroscience 13, 1428-1432. (2010).

What is claimed is:

1. An apparatus for managing a biological substrate, said apparatus comprising:
means of creating a counterpart environment to the biological substrate;
means of operatively connecting said counterpart environment to said biological substrate;
means of characterizing said biological substrate on the basis of the modulation of said counterpart environment by said biological substrate;
means of utilizing said characterization of said biological substrate.

2. The apparatus of claim 1 wherein said counterpart environment comprises a sensor ensemble disposed on an arbitrarily shaped and arbitrarily sized N-dimensional surface wherein each sensor is connected operatively to a suitable medium which in turn is connected operatively to the biological substrate; wherein said sensors are responsive to stimuli from said biological substrate.

3. The apparatus of claim 1 wherein said medium is a conducting medium.

4. The apparatus of claim 1 wherein said characterization of said biological substrate involves the determination of at least one state of said biological substrate.

5. The apparatus of claim 1 wherein said utilization of said characterization of said biological substrate involves translation of at least one state of said biological substrate into at least one command that may be used to control any aspect of an external system such as the position of a cursor in a computer system.

6. An apparatus for managing a biological substrate, said apparatus comprising:
means of creating a counterpart environment to the biological substrate;
means of operatively connecting said counterpart environment to said biological substrate;
means of modulating said biological substrate by modulating said counterpart environment.

7. The apparatus of claim 6 wherein said counterpart environment comprises an actuator ensemble disposed on an arbitrarily shaped and arbitrarily sized N-dimensional surface wherein each actuator is connected operatively to a suitable medium which in turn is connected operatively to the biological substrate; wherein said actuators generate suitable stimuli within said biological substrate.

8. The apparatus of claim 6 wherein said medium is a conducting medium.

9. The apparatus of claim 6 wherein said modulation of said biological substrate involves the modulation of at least one state of said biological substrate.

10. The apparatus of claim 6 wherein said modulation of said biological substrate results in the acquisition of at least one desired characteristic by said biological substrate.

11. An apparatus for managing a biological substrate, said apparatus comprising:
means of creating a counterpart environment to the biological substrate;
means of operatively connecting said counterpart environment to said biological substrate;
means of characterizing said biological substrate on the basis of the modulation of said counterpart environment by said biological substrate;
means of modulating said biological substrate by modulating said counterpart environment
means of moderating said modulation of said biological substrate on the basis of said characterization of said biological substrate.

12. The apparatus of claim 11 wherein said counterpart environment comprises a transducer ensemble disposed on an arbitrarily shaped and arbitrarily sized N-dimensional surface wherein each transducer is connected operatively to a suitable medium which in turn is connected operatively to the biological substrate; wherein said transducers are responsive to stimuli from said biological substrate when configured as sensors and generate stimuli within said biological substrate when configured as actuators.

13. The apparatus of claim 11 wherein said medium is a conducting medium.

14. The apparatus of claim 11 wherein said characterization of said biological substrate involves the determination of at least one state of said biological substrate.

15. The apparatus of claim 11 wherein said modulation of said biological substrate involves the modulation of at least one state of said biological substrate.

16. A method for managing a biological substrate, comprising steps of:
creating a counterpart environment to the biological substrate;
operatively connecting said counterpart environment to said biological substrate;
characterizing said biological substrate on the basis of the modulation of said counterpart environment by said biological substrate;
utilizing said characterization of said biological substrate.

17. The method of claim 16 wherein said counterpart environment comprises a sensor ensemble disposed on an arbitrarily shaped and arbitrarily sized N-dimensional surface wherein each sensor is connected operatively to a suitable medium which in turn is connected operatively to the biological substrate; wherein said sensors are responsive to stimuli from said biological substrate.

18. The method of claim 16 wherein said medium is a conducting medium.

19. The method of claim 16 wherein said characterization of said biological substrate involves the determination of at least one state of said biological substrate.

20. The method of claim 16 wherein said utilization of said characterization of said biological substrate involves translation of at least one state of said biological substrate into at least one command that may be used to control any aspect of an external system such as the position of a cursor in a computer system.

21. A method for managing a biological substrate, comprising steps of:
creating a counterpart environment to the biological substrate;
operatively connecting said counterpart environment to said biological substrate;
modulating said biological substrate by modulating said counterpart environment.

22. The method of claim 21 wherein said counterpart environment comprises an actuator ensemble disposed on an arbitrarily shaped and arbitrarily sized N-dimensional surface wherein each actuator is connected operatively to a suitable medium which in turn is connected operatively to the biological substrate; wherein said actuators generate suitable stimuli within said biological substrate.

23. The method of claim 21 wherein said medium is a conducting medium.

24. The method of claim 21 wherein said modulation of said biological substrate involves the modulation of at least one state of said biological substrate.

25. The method of claim 21 wherein said modulation of said biological substrate results in the acquisition of at least one desired characteristic by said biological substrate.

26. A method for managing a biological substrate, comprising steps of:
creating a counterpart environment to the biological substrate;
operatively connecting said counterpart environment to said biological substrate;
characterizing said biological substrate on the basis of the modulation of said counterpart environment by said biological substrate;
modulating said biological substrate by modulating said counterpart environment moderating said modulation of said biological substrate on the basis of said characterization of said biological substrate.

27. The method of claim 26 wherein said counterpart environment comprises a transducer ensemble disposed on an arbitrarily shaped and arbitrarily sized N-dimensional surface wherein each transducer is connected operatively to a suitable medium which in turn is connected operatively to the biological substrate; wherein said transducers are responsive to stimuli from said biological substrate when configured as sensors and generate stimuli within said biological substrate when configured as actuators.

28. The method of claim 26 wherein said medium is a conducting medium.

29. The method of claim 26 wherein said characterization of said biological substrate involves the determination of at least one state of said biological substrate.

30. The method of claim 26 wherein said modulation of said biological substrate involves the modulation of at least one state of said biological substrate.
